(19) United States
(12) Patent Application Publication
(10) Pub. No.: US 2016/0310328 A1
(43) Pub. Date: Oct. 27, 2016

(54) SWAB WITH ANNULAR GROOVES

(71) Applicant: Filo America, Commerce, CA (US)

(72) Inventors: Fred Pourbaba, Los Angeles, CA (US); Oliver Nathan Pourbaba, Los Angeles, CA (US)

(73) Assignee: Filo America

(21) Appl. No.: 14/695,911

(22) Filed: Apr. 24, 2015

(51) Int. Cl. A61F 13/38; A61F 11/00

(52) U.S. Cl. CPC A61F 13/38; A61F 11/006; A61F 2013/4587

(57) ABSTRACT

Swabs, according to the present invention, include a stern and at least one tip at an end of the stem and having a generally cylindrical absorbent material formed thereon. The tip has at least one annular groove. Preferably, the swab includes a plurality of equally spaced annular grooves to provide multiple sections of the swab for application or removal of material. By providing one or more spaced-apart annular grooves in the tip, material can be added or removed from a surface in a much more controlled fashion. By providing a tip with a substantially cylindrical shape with annular grooves, the swabs also have a unique appearance as compared to typical tapered swabs.

### BACKGROUND OF THE INVENTION

The present invention relates to swabs and, particularly, cotton swabs which can be used for a variety of medical and other purposes.

The manufacturing of conventional cotton swabs is well known. A swab applicator consists of a longitudinal stem which has a tip of soft and absorbent cotton or other materials typically wrapped or formed around one or both ends of the stem. Such applicator devices and mechanism for making the same are disclosed in U.S. Pat. Nos. 1,652,108; 1,721,815; 1,921,604; 1,997,467; 2,006,539; 2,090,354; 2,261,058; 2,430,648; 2,705,009; 2,987,063; and 4,707,318. In making a conventional swab of absorbent cotton or other material, it is well known to bond at least one end of a stem with a thin wad or strip of absorbent cotton using an aqueous adhesive. The wad is then wound or molded around the stem.

Swabs have been employed in a variety of applications including application of antiseptic material to wounds, cleaning wax from one's ear canal, and numerous other usual functions. Typically, the tips of a swab have a somewhat oval shape and are attached to a stem made of a polymeric or fibrous material. The tips of the swab typically are made of cotton or similar absorbent material, which is applied to the ends of the stem utilizing a mold, calendar rollers, or the like.

The use of such swabs for application of material to one's skin or for cleaning one's ear canal is well known using a conventionally formed oval cotton swab. Swabs are frequently also used for non-medical purposes. Although some improvements to the basic form of cotton swabs has been suggested, Applicant has discovered that by providing one or more annular grooves along the length of the tip of the swab, which has a generally cylindrical shape, has proven effective not only for use in cleaning an ear canal but also for controlling the application of liquids, salves and other material on a person's skin or on a wound.

### SUMMARY OF THE INVENTION

Swabs, according to the present invention, include a stem and at least one tip at an end of the stem and having a generally cylindrical absorbent material formed thereon. The tip has at least one annular groove. Preferably, the swab includes a plurality of equally spaced annular grooves to provide multiple sections of the swab for application or removal of material. By providing one or more spaced-apart annular grooves in the tip, material can be added or removed from a surface in a much more controlled fashion. By providing a tip with a substantially cylindrical shape with annular grooves, the swabs also have a unique appearance as compared to typical tapered swabs.

These and other features, objects and advantages of the present invention will become apparent upon reading the following description thereof together with reference to the accompanying drawings.

### BRIEF DESCRIPTION OF THE DRAWINGS

FIG. 1 is a perspective view of a first embodiment of the present invention;

FIG. 2 is a side elevational view of the swab shown in FIG. 1;

FIG. 3 is an end elevational view of the swabs shown in all of the figures;

FIG. 4 is a perspective view of an alternative embodiment of the swab using a single annular groove;

FIG. 5 is a side elevational view of the swab shown in FIG. 4;

FIG. 6 is a perspective view of another embodiment of the swab using two annular grooves;

FIG. 7 is a side elevational view the swab shown in FIG. 6;

FIG. 8 is a perspective view of a swab using four annular grooves; and

FIG. 9 is a side elevational view of the swab shown in FIG. 8.

### DETAILED DESCRIPTION OF THE PREFERRED EMBODIMENT

The swab of the present invention can be manufactured using the conventional techniques discussed above, however, it is finish molded into the unique shape shown in the attached figures to include one or a plurality of spaced-apart annular grooves in a generally cylindrically shaped tip body.

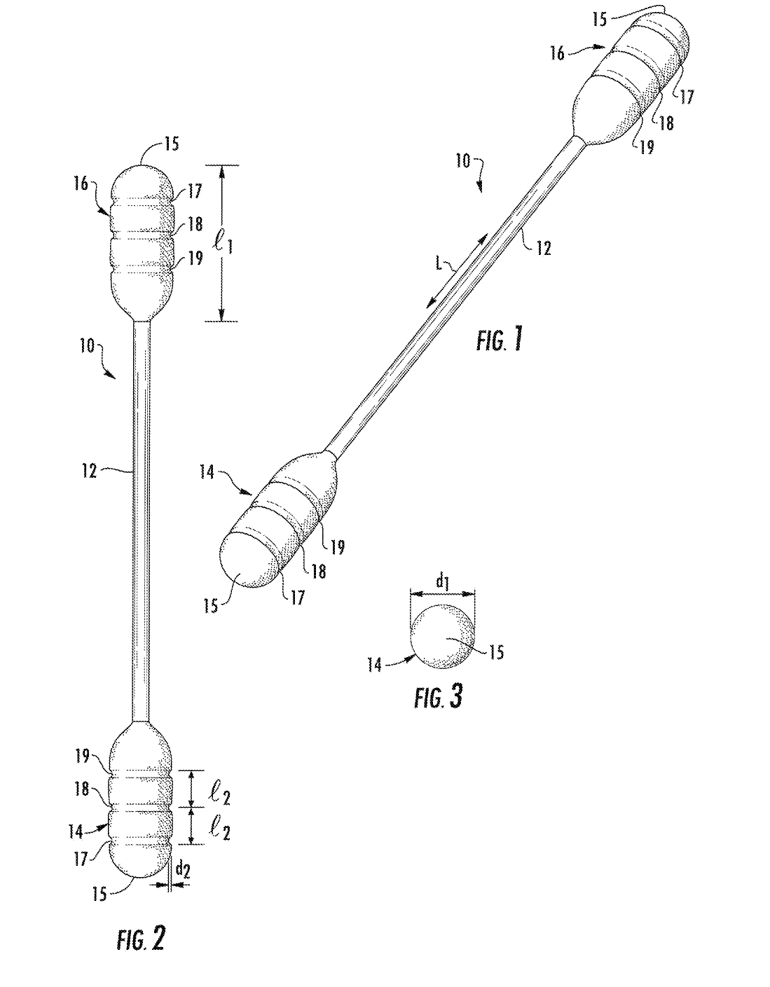

Referring initially to FIGS. 1-3, there is shown a swab 10 of a first embodiment of the invention comprising a elongated cylindrical stem 12, which can be made of a thermoplastic material, a fibrous material, or other suitable materials typically employed for the manufacture of swabs. Swab 10 includes tips 14 and 16 at opposite ends of the stem 12. In some embodiments, the swab will include a single tip at one end. Each of the tips 14 and 16 are substantially identical and comprise a generally cylindrical body as viewed along the longitudinal axis “L” (FIG. 1) of the swab 10. Each tip includes a rounded hemispherical end 15 and a plurality of longitudinally spaced-apart annular grooves 17, 18, and 19. The fibrous absorbent materials employed for the tips 14 and 16 can be white or any desired color, such as pink, blue, green or the like, as desired, for possible identification for use for separate functions.

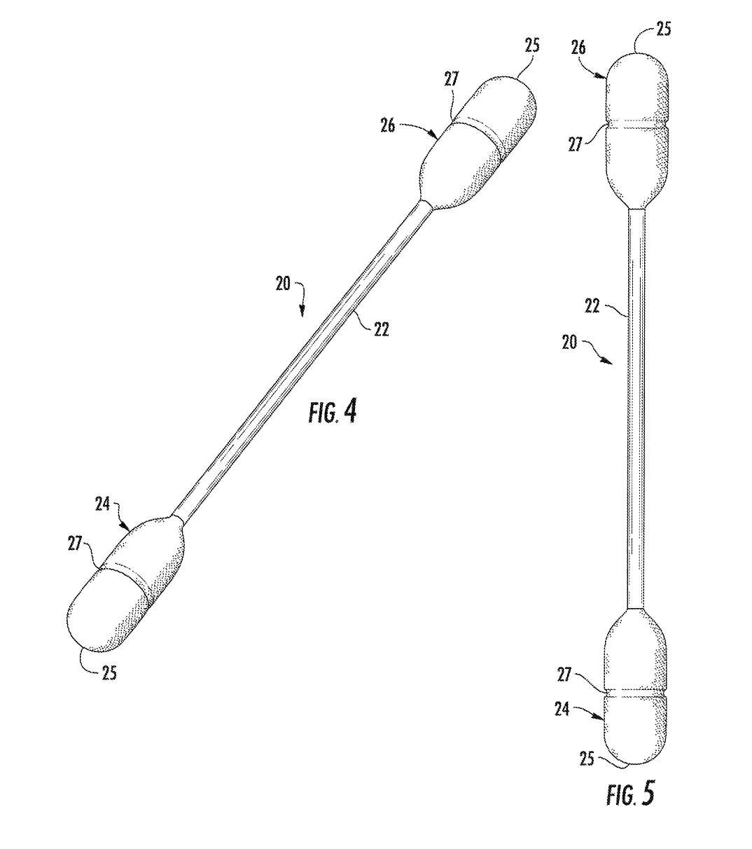

In the embodiment shown in FIGS. 1-3, the swab 10 has a length of about 3″ with each of the tips 14, 16 having a length l₁ of about 0.5″ and an outer diameter d1 (FIG. 3) of about ¼″. The depth d2 (FIG. 2) of each of the annular grooves is about 1/32″ (FIG. 2). The grooves are spaced apart from one another a distance l₂ of approximately 1/10″ in the embodiment of FIGS. 1-3. The radius of curvature of ends 15 is about 25 mm. These dimensions can be varied within +/− 10% to still maintain an effective swab for application or removal of material onto or from a surface.

FIGS. 4 and 5 show an alternative embodiment of a swab 20 which includes a stem 22 of the same material as in the prior embodiment and tips 24, 26. The tips 24, 26 each include a single groove 27 longitudinally centered in the tip as opposed to the multiple grooves of the first embodiment. The dimensions of the tips are otherwise substantially identical, and the rounded ends 25 are substantially the same shape and dimensions as shown in FIG. 3.

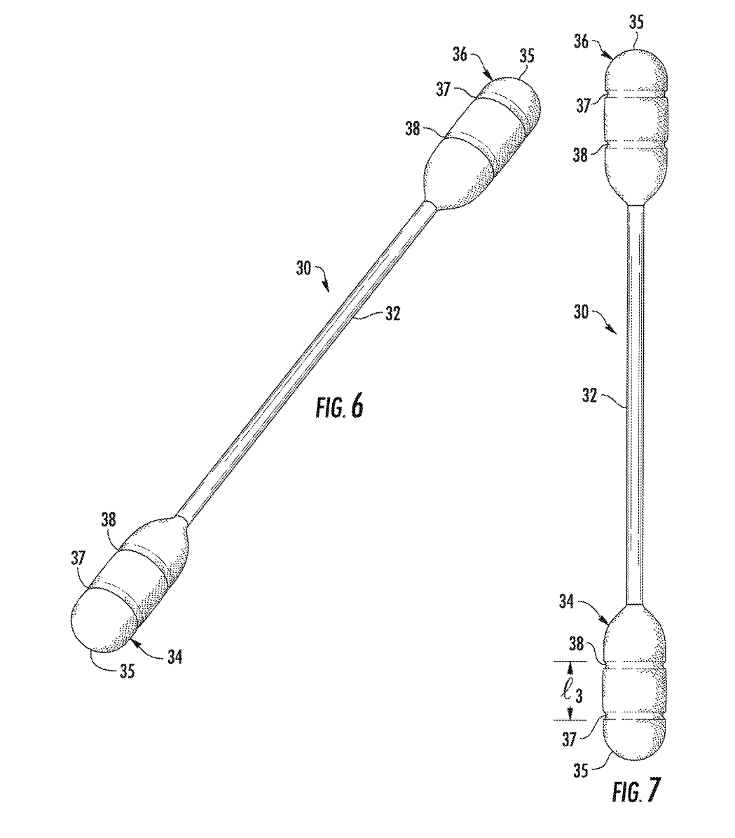

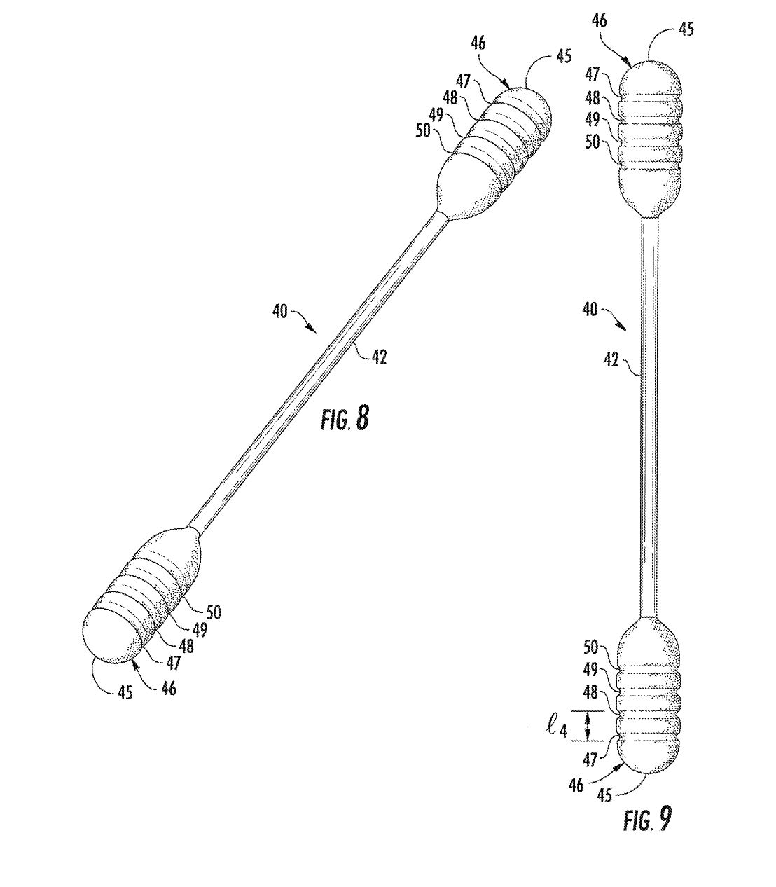

FIGS. 6 and 7 show yet another embodiment of a swab. Swab 30 includes a stem 32 of the same material as in the prior embodiments. Swab 30 includes tips 34, 36 with ends 35 having the same appearance and shape as ends 15 in the first embodiment and shown in FIG. 3. The material of tips 34, 36 likewise is the same as can be its coloration. Tips 34, 36, however, include a pair of spaced-apart grooves 37, 38 as opposed to a single groove as in the prior embodiment. Grooves 37, 38 are spaced apart a spacing l₃ between grooves 37 and 38 which is about 0.2″. The swab of FIGS. 6 and 7 otherwise has the same dimensions as the swab of the first embodiment shown in FIGS. 1-3.

FIGS. 8 and 9 show yet another embodiment of a swab 40 having a stem 42 and tips 44, 46, each with hemispherical end 45 of the same shape and dimension as end 15 of the FIGS. 1-3 embodiment. In the embodiment shown in FIGS. 8 and 9, the tips include four annular grooves 47, 48, 49, and 50, which are equally longitudinally spaced from one another along the longitudinal axis of the swab 40. Spacing l₃ between the grooves in the embodiment shown in FIGS. 8 and 9 is approximately 0.075″. The dimensions of the tips of all of the embodiments are substantially the same and differ only in the number of grooves.

It will become apparent to those skilled in the art that various modifications to the preferred embodiment of the invention as described herein can be made without departing from the spirit or scope of the invention as defined by the appended claims.

The invention claimed is:

1. A swab comprising:
a stem; and
a tip of absorbent material attached to at least one end of said stem, said tip having a generally cylindrical shape and including at least one external annular groove formed therein.

2. The swab as defined in claim 1 wherein said tip includes a plurality of longitudinally spaced annular grooves.

3. The swab as defined in claim 1 wherein said tip includes a pair of longitudinally spaced annular grooves.

4. The swab as defined in claim 1 wherein said tip includes three longitudinally spaced annular grooves.

5. The swab as defined in claim 1 wherein said tip includes four longitudinally spaced annular grooves.

6. The swab as defined in claim 1 wherein said stem includes a tip of absorbent material attached at opposite ends and each tip includes one or more external annular grooves formed therein.

7. A swab comprising:
a stem; and
a tip of absorbent material attached to opposite ends of said stem, said tips having a plurality of longitudinally spaced external annular grooves formed therein.

8. The swab as defined in claim 7 wherein said stem is made of a polymeric material.

9. The swab as defined in claim 7 wherein said stem is made of a fibrous material.

10. The swab as defined in claim 7 wherein said tip is made of a material including cotton.

11. The swab as defined in claim 7 wherein said tips are a color other than white.

12. The swab as defined in claim 11 wherein said tips are pink.

13. A swab comprising:
a stem; and
a generally cylindrical tip of absorbent material attached to at least one end of said stem, said tip having a plurality of longitudinally spaced inwardly extending annular grooves formed therein.

14. The swab as defined in claim 13 wherein said swab includes a tip at opposite ends of said stem.

15. The swab as defined in claim 13 wherein said stem is made of a polymeric material.

16. The swab as defined in claim 13 wherein said tip includes a pair of longitudinally spaced annular grooves.

17. The swab as defined in claim 13 wherein said tip includes three longitudinally spaced annular grooves.

18. The swab as defined in claim 14 wherein said tip includes four longitudinally spaced annular grooves.

19. The swab as defined in claim 13 wherein said tip is made of a material including cotton.

20. The swab as defined in claim 13 wherein said tip is pink.
